(19) United States
(12) Patent Application Publication
(10) Pub. No.: US 2007/0099850 A1
(43) Pub. Date: May 3, 2007

(54) Combination of acetylsalicylic acid and alpha-glucosidase inhibitors

(75) Inventors: Gilbert Wagener, Unna (DE); Khairy Elmahdi, Frechen (DE); Uwe Hohner, Langenfeld (DE)

Correspondence Address: JEFFREY M. GREENMAN, BAYER PHARMACEUTICALS CORPORATION, 400 MORGAN LANE, WEST HAVEN, CT, 06516, US

(73) Assignee: BAYER HEALTHCARE AG, GERMANY (DE)

(21) Appl. No.: 10/557,264

(22) PCT Filed: May 15, 2004
(86) PCT No.: PCT/EP04/05253

§ 371 Date: Jan. 18, 2007

(30) Foreign Application Priority Data
May 28, 2003 (DE) 103-24-282.1

(51) Int. Cl. A61K 31/7034; A61K 31/60

(52) U.S. Cl. 514/25; 514/165

(57) ABSTRACT

The invention relates to a combination of acetylsalicylic acid (component A) with an alpha-glucosidase inhibitor (component B) for the prevention of cardiovascular disorders.

The invention relates to a combination of acetylsalicylic acid (component A) with an alpha-glucosidase inhibitor (component B) for the prevention of cardiovascular disorders.

The compound of component A is well known to the skilled person. It is acetylsalicylic acid which is employed in particular for the secondary (Antiplatelet Trialists' Collaboration: Collaborative overview of randomised trials of antiplatelet therapy. I: Prevention of death, myocardial infarction, and stroke by prolonged antiplatelet therapy in various categories of patients. Brit. Med. J. 1994, 308, 81-106) but also for the primary prevention of cardiovascular disorders (U.S. Preventive Services Task Force: Aspirin for the primary prevention of cardiovascular events: recommendation and rationale. Ann. Intern. Med. 2002, 136, 157-160). A significantly beneficial effect in the primary prevention of cardiovascular disorders is observed specifically on treatment of hypertensive patients with a blood pressure-lowering agent on additional administration of acetylsalicylic acid (Effects of intensive blood-pressure lowering and low-dose aspirin in patients with hypertension: principal results of the Hypertension Optimal Treatment (HOT) randomised trial, THE LANCET 351, 1998, 1755-1762).

Compounds of component B are known to the skilled person as substances regulating the blood glucose level. They are alpha-glucosidase inhibitors, of which acarbose in one. As one of the standard therapies for treating type 2 diabetes mellitus, acarbose is described in many publications and textbooks (Leboritz, et. al, alpha-Glucosidase inhibitors as agents in the treatment of diabetes, Diabetes Reviews 1998; 6(2): 132-45).

There is no reference to a combination of acetylsalicylic acid (component A) with an alpha-glucosidase inhibitor (component B) for the prevention of cardiovascular disorders.

The present invention relates to the combination of acetylsalicylic acid as component A with an alpha-glucosidase inhibitor as component B for the prevention of cardiovascular disorders.

Prevention means both primary and secondary prevention. Primary prevention means in this context protecting patients from a first cardiovascular disorder resulting in organ damage. Secondary prevention means in this context protecting patients who have already suffered organ damage as a result of a cardiovascular disorder from a new cardiovascular disorder.

The invention further relates to pharmaceutical preparations comprising these combinations of A and B and the production thereof.

Alpha-glucosidase inhibitors for the purposes of the invention are in general all substance classes and substances included within this concept in the state of the art, such as, for example, acarbose, miglitol and voglibose. Acarbose is preferred within this concept.

The present invention preferably relates to the combination of acetylsalicylic acid as component A with an alpha-glucosidase inhibitor as component B for the prevention of cardiovascular disorders in patients with an increased risk of suffering from a cardiovascular disorder.

In patients who have an increased risk of suffering a cardiovascular disorder, the combinations according to the invention show an unexpected broad and diverse range of effects.

The group of patients mentioned herein with an increased risk includes, for example, patients with elevated blood pressure (hypertension), patients with a risk of developing high blood pressure, patients with disturbances of lipid metobolism such as, for example, hyperlipidaemia or dyslipidaemia, patients with renal dysfunction such as, for example, mild renal heart failure (MRF), having an elevated plasma creatinine level, patients with diabetes, patients with type 2 diabetes (non-insulin-dependent diabetes mellitus/NIDDM), patients with impaired glucose metabolism (prediabetic state), patients with an increased body mass index (BMI), patients with first-degree relatives who are suffering or have suffered from a cardiovascular disorder, and patients with first-degree relatives who are suffering or have suffered from diabetes. An elevated creatinine level is present in particular when the level is above 1.5 mg/dl in men and 1.4 mg/dl in women. An increased BMI is present in particular when the value is above 25 kg/m². Patients with hypertension are at increased risk especially when the total cholesterol level is above 200 mg/dl and/or the LDL level (low density lipoprotein) is above 160 mg/dl. Patients with diabetes, a prediabetic state or renal dysfunction are at increased risk especially when the total cholesterol level is above 170 mg/dl and/or the LDL level (low density lipoprotein) is above 120 mg/dl. Patients with elevated blood pressure include in particular patients with only slightly elevated blood pressure (120/85 mm-Hg to 139/90 mmHg) and patients whose existent elevated blood pressure has undergone an insufficient reduction in the elevated blood pressure (BP>145/95 mmHg).

“Hyperlipidaemia” is intended to mean an elevated plasma level of one or more serum lipids. The LDL level is particularly important in this connection. Levels above 130 mg/dl in patients of over 45 years of age and levels above 160 mg/dl in patients below 45 years of age are regarded as elevated levels.

“Dyslipidaemia” is intended here to mean either a hypertriglyceridaemia or a hypercholesterolaemia, but especially a mixed hyperlipidaemia, i.e. a pathological state with elevated cholesterol level (LDL and total cholesterol) and elevated triglyceride level. This may be associated with a reduction in the HDL (high density lipoprotein) cholesterol in the plasma or a stored HDL-C/LDL-C ratio.

The group of patients mentioned herein with an increased risk includes, particularly preferably patients with renal dysfunction such as, for example, mild renal heart failure (MRF), having an elevated plasma creatinine level, patients with disturbances of lipid metabolism such as hyperlipidaemia, dyslipidaemia, patients with elevated blood pressure (hypertension), patients with impaired glucose metabolism (prediabetic state), or patients with first-degree relatives who are suffering or have suffered from diabetes, without these patients having diabetes.

Cardiovascular disorders mean disorders such as arteriosclerosis, stroke, angina pectoris, disorders of the coronary vessels of the heart, especially of the arterial coronary vessels, heart failure, primary myocardial infarction, pathological changes in the vessel wall, circulatory disturbances, microcirculatory disturbances, disturbances of lipid metabolism such as hyperlipidaemia, dyslipidaemia, elevated serum lipoprotein concentration and possibly a shift in the lipoprotein fractions, hyperlipoproteinaemia, elevation both of serum cholesterol and of serum triglycerides combined with elevated VLDL (very low density lipoprotein) and elevation of chylomicrons in the plasma, non-insulin-dependent diabetes mellitus (=type 2 diabetes), diabetes, hyperglycaemia, metabolic disturbances such as disturbance of lipid metabolism, deficiency of acid lipase, storage diseases, especially lipid storage diseases, phytosterolaemia, high blood pressure (hypertension), obesity, thromboses, pancreatitis, constipation, functional disorders of the brain, cerebrovascular insufficiency, cerebral blood flow disorders, stroke, transient ischaemic attacks (TIA) and syncope.

The combinations according to the invention prove to be surprisingly advantageous in the prevention of coronary heart disease, heart failure, cognitive impairment, stroke, circulatory disturbances, non-insulin-dependent diabetes mellitus (=type 2 diabetes), diabetes, disturbances of glucose metabolism or high blood pressure (hypertension) and especially in the prevention of patients at increased risk of suffering a cardiovascular disorder.

The combinations according to the invention in particular prove to be surprisingly advantageous in the prevention of non-insulin-dependent diabetes mellitus (=type 2 diabetes) or diabetes.

There is particular interest in the use of the combinations according to the invention in so-called cardiac risk management, i.e. in the prophylaxis of cardiovascular disorders which are influenced or caused by more than one risk factor, such as, for example, arteriosclerosis, disorders of the coronary vessels of the heart, especially the arterial coronary vessels, elevated serum lipids, hypercholesterolaemia, hypertriglyceridaemia, elevation both of serum cholesterol and of serum triglycerides combined with elevated VLDL (very low density lipoprotein) and elevation of the chylomicrons in the plasma and syndrome X. Typical risk factors are elevated cholesterol level, reduced HDL level, smoking, glucose intolerance and enlargement of the heart. The risk factors may be different depending on the age and sex of the patient.

On use of the combinations according to the invention, a synergistic effect which was not to be expected was observed in the action. It is thus possible to reduce the amounts employed of components A and B compared with the monotherapy.

The effect is particularly pronounced in patients with renal dysfunction such as, for example, mild renal heart failure (MRF) who have an elevated plasma creatinine level, patients with disturbances of lipid metabolism such as, for example, hyperlipidaemia or dyslipidaemia, patients with elevated blood pressure (hypertension) or patients with impaired glucose metabolism (prediabetic state), without these patients having diabetes.

It may be expedient where appropriate to supplement the combinations according to the invention by adding one or more further components. Examples which may be mentioned are vitamin C, vitamin E and L-arginine. These other components may be added singly or else together.

The combinations according to the invention are further distinguished by a surprisingly good tolerability.

The combinations according to the invention are preferably employed in human medicine but are also suitable for veterinary medicine, especially for the treatment of mammals.

Administration of the combinations according to the invention can take place parenterally or preferably orally.

“Combinations” mean for the purposes of the invention not only dosage forms which contain both components (so-called fixed combinations), and combination packs containing the components separate from one another, but also components which are administered simultaneously or sequentially, as long as they are employed for the prophylaxis or treatment of the same disease.

The active ingredients of components A and B can be converted in a known manner into the usual formulations, which may be liquid or solid formulations. Examples are tablets, coated tablets, pills, capsules, granules, aerosols, syrups, emulsions, suspensions, solutions.

Since the combinations according to the invention are well tolerated and are effective even in low dosages, a wide range of formulation variants is possible. Thus, one possibility is to formulate the individual components separately. In this case, it is not absolutely necessary for both individual components A and B to be taken at the same time; on the contrary, sequential intake may be advantageous to achieve optimal effects. It is appropriate with such separate administration to combine the formulations of both individual components, for example tablets or capsules, simultaneously together in a suitable primary packaging. Both components are present in the primary packaging in each case in separate containers which may be, for example, tubes, bottles or blister packs. Such separate packaging of both components in the joint primary packaging is also referred to as a kit.

Further formulation variants which are suitable and preferred for the combinations according to the invention are also fixed combinations. “Fixed combination” is intended here to mean pharmaceutical forms in which both components are present together in a fixed ratio of amounts. Such fixed combinations may be, for example, in the form of oral solutions, but they are preferably solid oral pharmaceutical preparations, e.g. capsules or tablets.

The combinations according to the invention are given up to 3×a day, preferred combinations being those permitting administration 1×+2×a day.

The combinations according to the invention preferably contain 0.01 to 20 mg/kg, in particular 0.1 to 5 mg/kg, of active ingredient of component A and 0.01 to 20 mg/kg, in particular 0.1 to 5 mg/kg, of active ingredient of component B, in each case based on kg of the patient's bodyweight on oral administration.

The synergistic effect of the combinations according to the invention is furthermore preferably observed when the combinations according to the invention contain as component A acetylsalicylic acid in dosages of 5 to 500 mg, preferably in dosages of 50 to 350 mg, particularly preferably in a dosage of 100 mg, and as component B acarbose in dosages of 5 to 500 mg, preferably in dosages of 30 to 350 mg, particularly preferably in a dosage of 50 or 100 mg.

The synergistic effect of the combinations according to the invention is preferably observed when components A and B of the combinations according to the invention are present in a ratio of 1:10 to 10:1, preferably 1:5 to 5:1, particularly preferably 1:2 to 2:1, in relation to A and B.

It may where appropriate be necessary to deviate from the stated amounts, in particular depending on the bodyweight or the nature of the administration route, on the individual behaviour towards the medicament, the nature of the formulation thereof and the time or interval over which administration takes place. Thus, it may be sufficient in some cases to make do with less than the aforementioned minimum amount, whereas in other cases the upper limit mentioned must be exceeded. It may be advisable where relatively large amounts are administered to divide these into a plurality of single doses over the day.

The active ingredients of components A and B are particularly suitable for formulation in a fixed combination in the form of a solid oral dosage form. It is generally known that the factors on which the patients' reliability of intake (compliance) crucially depend are the number of dosage forms per time of intake and the size and weight of the (solid oral) pharmaceutical form. Hence both the number of the different medicaments to be taken separately should be as small as possible (advantage of a fixed combination), and the size and weight of a solid oral dosage form should be as small as possible while having full therapeutic potency, in order to make intake as pleasant as possible for the patient. It is thus possible to attain fixed combinations in the form of solid oral pharmaceutical formulations of minimal size and minimal weight. The fixed combinations according to the invention accordingly provide maximum patient compliance and thus crucially improve the safety and reliability of therapy.

The release of active ingredient can be controlled by combining both components A and B and modifying the composition or the functionality. For example, the abovementioned temporal uncoupling of the onset of action is possible even in fixed combinations through delayed release of active ingredient (slowing of release) of one component.

The solid oral dosage forms mentioned herein are produced by general standard processes. Ingredients are those which are pharmaceutically accepted and physiologically unobjectionable, for example: as fillers cellulose derivatives (e.g. microcrystalline cellulose), sugars (e.g. lactose), sugar alcohols (e.g. mannitol, sorbitol), inorganic fillers (e.g. calcium phosphates), binders (e.g. polyvinylpyrrolidone, gelatin, starch derivatives and cellulose derivatives), and all other excipients required to produce pharmaceutical formulations of the desired properties, e.g. lubricants (magnesium stearate), e.g. disintegrants (e.g. crosslinked polyvinylpyrrolidone, sodium carboxymethylcellulose), e.g. wetting agents (e.g. sodium lauryl sulphate), e.g. release-slowing agents (e.g. cellulose derivatives, polyacrylic acid derivatives), e.g. stabilizers, e.g. flavourings, e.g. coloured pigments.

Liquid formulations are likewise produced by a standard method using pharmaceutically usual excipients and contain the active ingredient or both active ingredients either dissolved or suspended. Typical administration volumes of these pharmaceutical preparations are 1 to 10 ml. Examples of excipients in these liquid formulations are: solvents (e.g. water, alcohol, natural and synthetic oils, e.g. medium chain-link triglycerides), solubilizers (e.g. glycerol, glycol derivatives), wetting agents (e.g. polysorbate, sodium lauryl sulphate), and further excipients required to produce pharmaceutical formulations of the desired properties, e.g. viscosity-increasing agents, e.g. pH-correcting agents, e.g. sweeteners and flavourings, e.g. antioxidants, e.g. stabilizers, e.g. preservatives.

The main ingredients of the shells of capsule formulations are, for example, gelatin or hydroxypropylmethylcellulose.

Pharmaceutical excipients familiar to the skilled person are also described for example in the following handbook: “Handbook of Pharmaceutical Excipients”, Wade, A. & Weller, P. J., American Pharmaceutical Association, Washington, 2nd edition 1994.

### EXEMPLARY EMBODIMENTS

### Example 1

Acetylsalicylic acid as component A in a dosage of 100 mg and acarbose as component B in a dosage of 50 mg.

### Example 2

Acetylsalicylic acid as component A in a dosage of 100 mg and acarbose as component B in a dosage of 75 mg.

### Example 3

Acetylsalicylic acid as component A in a dosage of 100 mg and acarbose as component B in a dosage of 100 mg.

### Exemplary Embodiments of Pharmaceutical Compositions

The compounds of the invention can be converted into pharmaceutical preparations in the following way:

Tablet:

Composition:

100 mg of the combination of Example 1, 50 mg of lactose (monohydrate), 50 mg of corn starch (native), 10 mg of polyvinylpyrolidone (PVP 25) (from BASF, Ludwigshafen, Germany) and 2 mg of magnesium stearate. Tablet weight 232 mg. Diameter 8 mm, radius of curvature 12 mm.

Production:

The mixture of active ingredients, lactose and starch is granulated with a 5% strength solution (m/m) of the PVP in water. The granules are then dried and mixed with the magnesium stearate for 5 min. This mixture is compressed using a conventional tablet press (see above for format of the tablet). A guideline for the compressive force used for the compression is 15 kN.

1. A combination comprising acetylsalicylic acid (component A) and an alpha-glucosidase inhibitor (component B).

2. (canceled)

3. (canceled)

4. (canceled)

5. A combination according to claim 1, characterized in that component A is acarbose.

6. A combination according to claim 1, characterized in that the combination contains 0.1 to 5 mg/kg active ingredient of component A and 0.1 to 5 mg/kg active ingredient of component B based on kg of the patient's bodyweight.

7. A combination according to claim 1, characterized in that the combination contains components A and B in a ratio of 1:2 to 2:1 relative to A and B.

8. A method for preventing a cardiovascular disorder, comprising administering to a patient a therapeutically acceptable amount of a combination of claim 1.

9. The method of claim 8, wherein the cardiovascular disorder is coronary heart disease, heart failure, cognitive dysfunction, stroke, circulatory disturbances, non-insulin-dependent diabetes mellitus (=type 2 diabetes), diabetes, stroke, disturbances of glucose metabolism or high blood pressure (hypertension).

10. The method of claim 9, wherein the cardiovascular disorder is non-insulin-dependent diabetes mellitus (=type 2 diabetes) or diabetes.

11. The method of claim 8, wherein the patient has an increased risk of suffering a cardiovascular disorder.

12. The method of claim 8, wherein the patient has renal dysfunction, mild renal heart failure (MRF) and an having an elevated plasma creatinine level, disturbances of lipid metabolism, hyperlipidaemia, dyslipidaemia, elevated blood pressure (hypertension), impaired glucose metabolism (prediabetic state), or first-degree relatives who are suffering or have suffered from diabetes, without said patient having diabetes.

13. A pharmaceutical composition comprising a combination according to claim 1 and where appropriate one or more further suitable components.

14. A process for producing a pharmaceutical composition according to claim 13, characterized in that the components A and B are converted with excipients and carriers and where appropriate with further components into a suitable administration form.

15. A kit comprising separate containers in a single package, comprising in one of said containers an effective amount of component A as defined in claim 1 or 5, in a pharmaceutically acceptable carrier, and in a second container an effective amount of component B as defined in claim 1 or 5, in a pharmaceutically acceptable carrier.
